(19) United States
(12) Patent Application Publication
(10) Pub. No.: US 2026/0096931 A1
(43) Pub. Date: Apr. 9, 2026

(54) MICRO ACTUATOR FOR INNER EAR INJECTION AND SAMPLING SURGERY

(71) Applicant: Harbin Institute of Technology, Harbin (CN)

(72) Inventors: He ZHANG, Harbin (CN); Haiming LI, Harbin (CN); Jie ZHAO, Harbin (CN)

(21) Appl. No.: 19/026,171

(22) Filed: Jan. 16, 2025

(30) Foreign Application Priority Data
Oct. 8, 2024 (CN) 202411393064.2

(51) Int. Cl. A61F 11/20; A61B 1/227; A61B 10/00

(52) U.S. Cl. CPC A61F 11/20; A61B 1/227; A61B 10/0045; A61B 2010/0054

(57) ABSTRACT

A micro actuator for inner ear injection and sampling surgery is provided. The microneedle puncture mechanism module is embedded in the driving and sensing module. The driving and sensing module is installed on the high-precision linear driving module. The end-effector mechanism module is installed in the front of the driving and sensing module. The microneedle puncture mechanism module passes through the end operating mechanism module and extends. An endoscope camera module located in the end-effector mechanism module provides real-time images of a surgical process. A tension sensor in the driving and sensing module monitors tension changes of steel wires in real time to ensure safe interaction with the intra-ear environment.

### TECHNICAL FIELD

The present invention relates to the field of medical apparatus and instruments, and particularly to a micro actuator for inner ear injection and sampling surgery.

### BACKGROUND

At present, inner ear dysfunction has a high incidence in the world, with symptoms such as hearing loss, tinnitus and vertigo, which often leads to serious sequelae and seriously affects the living quality of patients if not treated in time. Because the inner ear is located deeply in the temporal bone and has complex anatomical structure, traditional treatment methods such as systemic or local administration have limited efficacy, and invasive surgery such as cochlear implantation causes greater trauma. As a natural orifice of the human body, the ear canal provides a low-trauma path for the surgery. Through tympanic membrane perforation (which can be self-healing with a diameter less than 3 mm), the external auditory canal and the middle ear can be penetrated, providing a natural path for surgical instruments. Through round window membrane puncture, operations such as targeted drug injection and tissue fluid sampling of the inner ear can be carried out directly. Such method can effectively avoid use of traditional high-cost surgery and also reduce trauma and improve the therapeutic effect.

For example, in a patent for invention with an authorized announcement No. of CN116098762B and a name of an inner ear injection-sampling executor for an otologic surgical robot, the bottleneck that the blood labyrinth barrier of traditional inner ear administration hinders the accurate treatment of the inner ear is broken through, and the effective and controllable direct delivery of drugs to a target position is realized, greatly improving the surgical efficiency and efficacy of a drug delivery method; diseases and microenvironment changes are understood through tissue fluid sampling of the inner ear, which realizes early prevention and accurate diagnosis of inner ear diseases; and from the perspective of clinical surgery, the whole surgical process has a lower degree of injury to the human body, the time of surgery is shortened, and the surgical safety is greatly improved. A flexible segment module is used for the first time to realize the non-invasive operation of entering the target position of the inner ear round window via the natural orifice. The actuator can realize flexible four-degree-of-freedom bending motion at a micro-size (2 mm diameter), which effectively meets the rigid indexes of a narrow natural orifice surgical robot.

However, the executor does not exist alone in actual use. For example, during inner ear surgery, it is necessary for doctors to constantly pay attention to whether the end-effector mechanism makes contact with the intra-ear environment and judges the progress of the surgery while operating the robot for intervention. Therefore, it is necessary to cooperate to achieve accurate ear sampling and surgery. However, the existing actuators can see the operating environment of the inner ear through the endoscope, but cannot perceive the feedback during operation of the flexible segment module, which makes it unable to help the doctors judge the progress of the surgery more accurately, so that the otologic surgery is still difficult to operate. Moreover, the existing actuators realize the degree of freedom of overall feeding through the linear module, have a large size, and are not easy to integrate with a positioning robotic arm, thus lacking certain practicability.

To sum up, the existing actuators are unable to help the doctors judge the progress of the surgery more accurately so that the otologic surgery is still difficult to operate, and the existing actuators lack clinical surgery practicality in design and do not take a full account of the problems of system integration, surgical operation and disinfection treatment, which also brings some difficulties to surgical experiments and clinical promotion.

### SUMMARY

The purpose of the present invention is to solve the problem that the existing actuators are unable to help doctors judge the progress of surgery more accurately and lack clinical surgery practicality in design so that the otologic surgery is still difficult to operate. Then, a micro actuator for inner ear injection and sampling surgery is provided.

The technical solution of the present invention is as follows:

A micro actuator for inner ear injection and sampling surgery, comprising an end-effector mechanism module, a driving and sensing module, a microneedle puncture mechanism module and a high-precision linear driving module, wherein the driving and sensing module is installed on the high-precision linear driving module in a horizontal sliding manner and driven by the high-precision linear driving module to horizontally move, the microneedle puncture mechanism module is horizontally embedded in the driving and sensing module, the end-effector mechanism module is installed in the front of the driving and sensing module, the microneedle puncture mechanism module passes through the end-effector mechanism module and extends, an endoscope module located in the end-effector mechanism module provides real-time images of a surgical process, and a tension sensor in the driving and sensing module monitors tension changes of steel wires in real time to ensure safe interaction with the intra-ear environment and provide early warning information when necessary.

Further, the end-effector mechanism module comprises a plurality of rolling saddle segments, an endoscope module and a flexible microneedle, the rolling saddle segments are connected in series by multiple steel wires within the driving and sensing module, enabling multi-degree-of-freedom large-angle bending both in-plane and out-of-plane under the varying tension of each steel wire., the plurality of rolling saddle segments form a hollow flexible structure, the endoscope module and the flexible microneedle are embedded inside the flexible structure, and the endoscope module is located at the end of the flexible structure.

Further, the driving and sensing module comprises a plurality of driving units which are installed on a frame in a ring array; and each driving unit comprises a steel wire guiding mechanism, a tension sensor, a signal amplifier, a micro guide rail, a steel wire driving motor, a steel wire and an integrated driving board, the steel wire guiding mechanism is installed in the front of the frame, the steel wire driving motor is installed in the back of the frame, the integrated driving board is vertically installed at the rear end of the frame, the micro guide rail is installed in the middle of the frame, the tension sensor is fixedly installed on the micro guide rail by a slider, the signal amplifier is installed above the tension sensor, one end of the steel wire is connected with a threaded hole of the tension sensor by a wire drawing device, and the other end of the steel wire is fixed with the rolling saddle segments after bypassing the steel wire guiding mechanism.

Further, the microneedle puncture mechanism module comprises a plurality of needle feeding modules and a stepped shaft microneedle unit, the plurality of needle feeding mechanisms are axially installed inside the frame, and the microneedle unit is coaxially installed in a central hole of the needle feeding mechanisms.

Further, each needle feeding module comprises a head end protecting support, a needle feeding support, a microneedle puncture driving motor and a tail end fixing device, the needle feeding support and the microneedle puncture driving motor are installed on the frame and centrally located inside the driving and sensing module, an output shaft of the microneedle puncture driving motor drives a nut on a ball screw to make high-resolution linear motion and realize feeding motion through the needle feeding support fixedly connected therewith, and the head end protecting support is installed on the steel wire guiding mechanism to limit the maximum microneedle feeding displacement.

Further, the microneedle unit comprises a stepped shaft puncture needle and a Luer taper, the Luer taper is connected with the stepped shaft puncture needle, and the stepped shaft puncture needle passes through the head end protecting support and is connected with the head end protection support.

Preferably, the stepped shaft puncture needle is an integrated mechanism.

Furthermore, the micro actuator also comprises a quick-change mounting mechanism, and the quick-change mounting mechanism is installed on the lower end surface of the high-precision linear driving module and allows for quick attachment and detachment from an external robotic arm.

Furthermore, the micro actuator also comprises a split protective casing, and the split protective casing is encased over the end-effector mechanism module, the driving and sensing module, the microneedle puncture mechanism module and the high-precision linear driving module.

Preferably, the split protective casing comprises an actuating mechanism protective sleeve, an actuator casing and a linear driving casing, the actuating mechanism protective sleeve is sheathed on the end-effector mechanism module, the actuator casing is sheathed on the driving and sensing module and the microneedle puncture mechanism module, and the linear driving casing is sheathed on the high-precision linear driving module.

Compared with the prior art, the present invention has the following effects:

The present invention is a micro dexterous actuator for inner ear injection and sampling surgery. The actuator can realize minimally invasive operation through natural external auditory canal intervention, flexibly avoid obstacles in the ear under the visual guidance of the endoscope, and accurately reach the target region, thereby completing autonomous puncture injection and sampling operations. The present invention has the following advantages:
1. The present invention has the advantages of miniaturization and high flexibility: the present invention integrates the driving and sensing module 2 with the microneedle puncture mechanism module 3 in an embedded manner, and the length of the high-precision linear driving module 4 is greatly reduced so that the overall design size is smaller and the integration degree is higher, which can complete complex inner ear surgical procedures through the natural ear external auditory canal and meet the requirements of minimally invasive surgery.
2. The present invention has multi-functional integration: a variety of micro sensors are integrated, for example, a tension sensor and an endoscope, which work together to provide real-time images and force feedback, help the doctors judge the progress of the surgery, reduce the difficulty of operation and ensure surgical safety.
3. The present invention adopts modular and lightweight design: the modular structure design is adopted, the quick-change mounting mechanism 5 can facilitate easy attachment and detachment from the robotic arm, and the detachable split protective casing 6 can be disinfected in time, which fully consider the functional needs of surgery and clinical convenience.

### DESCRIPTION OF DRAWINGS

FIG. 1 is a physical diagram of the present invention;

FIG. 2 is a two-dimensional string diagram of an external structure of the present invention;

FIG. 3 is a main view of the present invention with a split protective casing 6 removed;

FIG. 4 is a main view of FIG. 3;

FIG. 5 is a top view of FIG. 3;

FIG. 6 is a local enlarged view of an end-effector mechanism module 1 of the present invention;

FIG. 7 is a bottom view of a quick-change mounting mechanism 5;

FIG. 8 is a top view of FIG. 7;

FIG. 9 is a flow block diagram of feeding back information by three forms of sensors adopted by the present invention to help doctors judge the progress of surgery more accurately;

FIG. 10 is an experimental diagram showing the puncture of the inner ear round window through the external auditory canal in human temporal bone and cadaveric specimens by the present invention.

### DETAILED DESCRIPTION

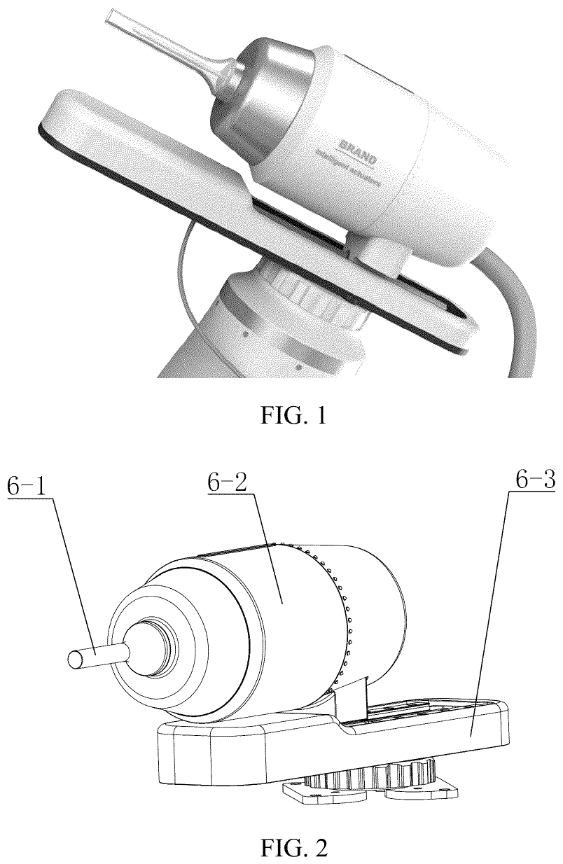

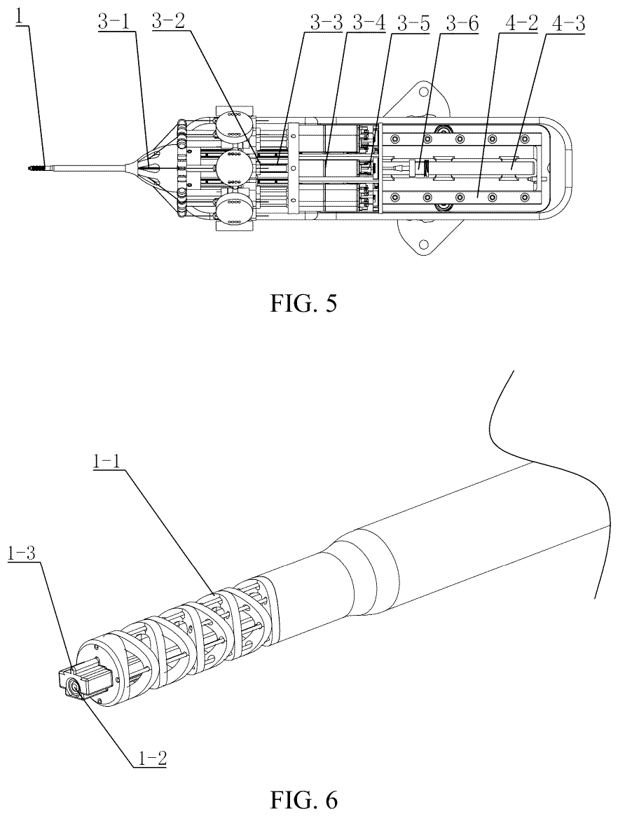

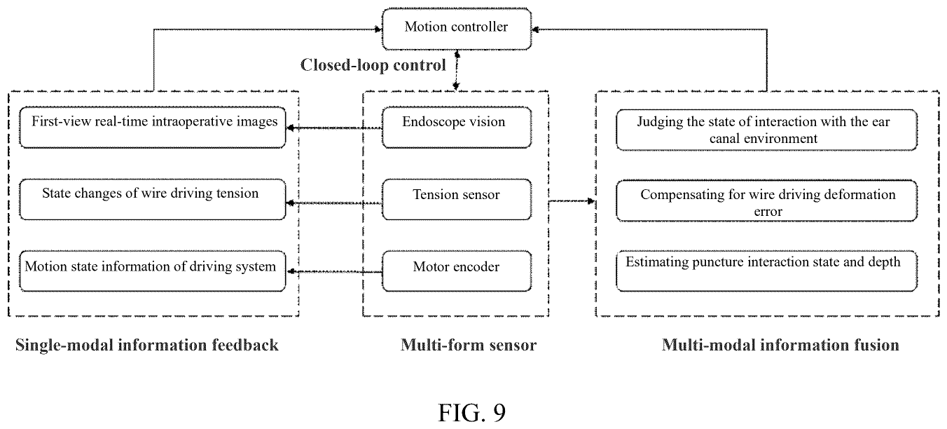

Specific embodiment 1: the present embodiment is described in conjunction with FIG. 1 to FIG. 9, and the present embodiment comprises an end-effector mechanism module 1, a driving and sensing module 2, a microneedle puncture mechanism module 3 and a high-precision linear driving module 4, wherein the driving and sensing module 2 is installed on the high-precision linear driving module 4 in a horizontal sliding manner and driven by the high-precision linear driving module 4 to horizontally move, the microneedle puncture mechanism module 3 is horizontally embedded in the driving and sensing module 2, the end-effector mechanism module 1 is installed at the front end of the driving and sensing module 2, the microneedle puncture mechanism module 3 passes through the end-effector mechanism module 1 and extends, an endoscope module 1-2 located in the end-effector mechanism module 1 provides real-time images of a surgical process, and a tension sensor 2-2 in the driving and sensing module 2 monitors tension changes of steel wires in real time to ensure safe interaction with an ear canal environment and provide early warning information when necessary.

On the premise of meeting the size requirements, the end-effector mechanism module 1 in the present embodiment adopts a hollow design, integrates saddle rolling segments, an endoscope, a light source, a puncture microneedle and other surgical tools, and also meets the needs of inner ear surgery for miniaturization, flexibility and multi-function of the actuating mechanism.

The high-precision linear driving module 4 in the present embodiment comprises a supporting base 4-1, a high-precision linear guide rail slider 4-2 and a linear feeding driving motor 4-3, the linear feeding driving motor 4-3 is horizontally installed on a linear driving casing 6-3, where it drives the high-precision linear guide rail slider 4-2 to horizontally slide, the supporting base 4-1 is installed on the high-precision linear guide rail slider 4-2, and the driving and sensing module 2 is installed on the supporting base 4-1.

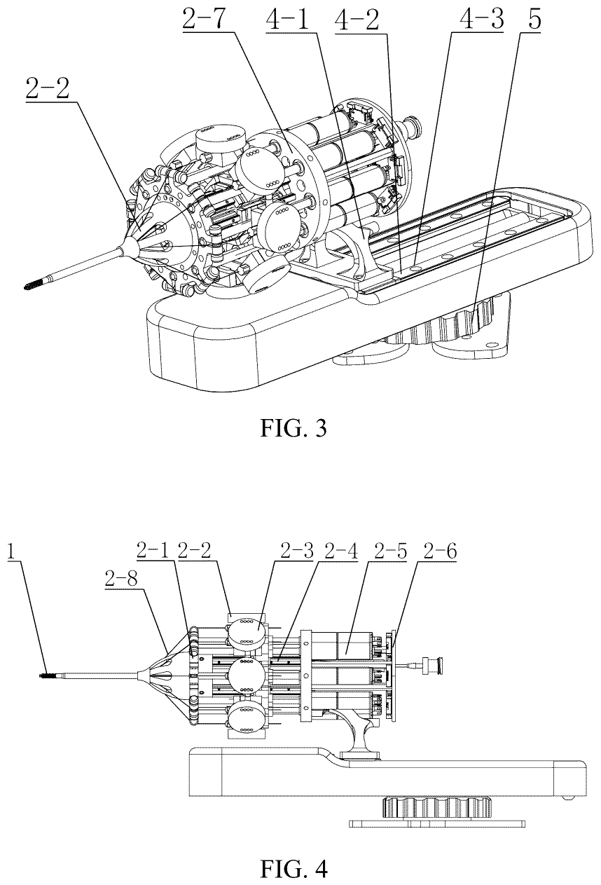

Specific embodiment 2: the present embodiment is described in conjunction with FIG. 3 to FIG. 6, and the end-effector mechanism module 1 in the present embodiment comprises a plurality of rolling saddle segments 1-1, an endoscope module 1-2 and a flexible microneedle 1-3, the rolling saddle segments 1-1 are connected in series by multiple steel wires within the driving and sensing module 2, enabling multi-degree-of-freedom large-angle bending both in-plane and out-of-plane under the varying tension of each steel wire, the plurality of rolling saddle segments 1-1 form a hollow flexible structure, the endoscope module 1-2 and the flexible microneedle 1-3 are embedded inside the flexible structure, and the endoscope module 1-2 is located at the end of the flexible structure.

In such arrangement, the flexible microneedle 1-3 which is a flexible metal microneedle is used for precise and safe puncture operations, thus realizing accurate drug delivery and sampling of the inner ear. The doctors can adjust the stretching and release of the driving wire through a remote control system to realize multi-degree-of-freedom bending motion of a multi-segmental flexible continuum mechanism composed of rolling segments. The endoscope and the light source provide high-definition real-time images for navigation. Other composition and connection relationships are the same as those in specific embodiment 1.

Compared with the existing continuum robot composed of saddle segments, the end-effector mechanism module 1 in the present embodiment is smaller in size and higher in integration specifically reflected as follows: the end-effector mechanism module 1 has an outer diameter of 2 mm and an inner diameter of only 1.3 mm, a two-section flexible mechanism (that specifically refers to the continuum part of the end-effector mechanism module 1, which is composed of saddle segments and divided into a proximal end and a distal end respectively with an independent bending degree of freedom and can realize the overall flexible bending motion of each section) and surgical instruments such as an endoscope and a microneedle are integrated at such a small dimension, and S-shaped four-degree-of-freedom bending can be realized at a length of only 7 mm, with a maximum bending angle of 150° and a minimum bending radius of only 0.8 mm.

Specific embodiment 3: the present embodiment is described in conjunction with FIG. 3 to FIG. 5, and the driving and sensing module 2 in the present embodiment comprises a plurality of driving units which are installed on a frame 2-7 in a ring array, wherein each driving unit comprises a steel wire guiding mechanism 2-1, a tension sensor 2-2, a signal amplifier 2-3, a micro guide rail 2-4, a steel wire driving motor 2-5, a steel wire 2-8 and an integrated driving board 2-6, the steel wire guiding mechanism 2-1 is installed in the front of the frame 2-7, the steel wire driving motor 2-5 is installed in the back of the frame 2-7, the integrated driving board 2-6 is vertically installed at the rear end of the frame 2-7, the micro guide rail 2-4 is installed in the middle of the frame 2-7, the tension sensor 2-2 is fixedly installed on the micro guide rail 2-4 by a slider, the signal amplifier 2-3 is installed above the tension sensor 2-2, one end of the steel wire 2-8 is connected with a threaded hole of the tension sensor 2-2 by a wire drawing device, and the other end of the steel wire 2-8 is fixed with the rolling saddle segments 1-1 after bypassing the steel wire guiding mechanism 2-1.

In such arrangement, the tension sensor 2-2 is arranged to feed back tension changes of each steel wire in real time, which, on one hand, can transmit information based on tension to compensate for the motion hysteresis error of the flexible section caused by tension deformation of the steel wire to further ensure the control accuracy, and on the other hand, can provide real-time feedback on the interaction between the surgical tool and the intra-ear environment, facilitating safe and accurate operation and avoiding the problems of secondary injuries to patients and unsuccessful surgery caused by inability to perceive the operation force. Other composition and connection relationships are the same as those in specific embodiment 1 or 2.

In the present embodiment, the integrated driving board 2-6 is connected with each motor, and control instructions are sent by a controller and a driver for signal transmission.

Specific embodiment 4: the present embodiment is described in conjunction with FIG. 3 to FIG. 5, and the microneedle puncture mechanism module 3 in the present embodiment comprises a plurality of needle feeding modules and a stepped shaft microneedle unit, wherein the plurality of needle feeding mechanisms are axially installed inside the frame 2-7, and the microneedle unit is coaxially installed in a central hole of the needle feeding mechanisms.

The driving and sensing module 2 in the present embodiment sends instructions to the motors through the integrated driving board 2-6, and utilizes transmission mechanisms such as a high-reduction gearbox, a ball screw and a linear guide rail to achieve precise control on displacement of the steel wires.

In this process, the steel wire guiding mechanism 2-1 is not only connected with the end-effector mechanism, but also keeps the motion direction of each steel wire stable and reduces the driving error.

The tension sensor 2-2 monitors tension changes of the steel wires in real time to ensure safe interaction with the intra-ear environment and provide early warning information when necessary. Such design effectively solves the limitation that a single endoscope cannot fully reflect the progress of the surgery, and provides more complete feedback for the surgical process.

Other composition and connection relationships are the same as those in any of specific embodiments 1-3.

Specific embodiment 5: the present embodiment is described in conjunction with FIG. 3 to FIG. 5, and each needle feeding module in the present embodiment comprises a head end protecting support 3-2, a needle feeding support 3-3, a microneedle puncture driving motor 3-4 and a tail end fixing device 3-5, wherein the needle feeding support 3-3 and the microneedle puncture driving motor 3-4 are installed on the frame 2-7 and centrally located inside the driving and sensing module 2, an output shaft of the microneedle puncture driving motor 3-4 drives a nut on a ball screw to make high-resolution linear motion and realize feeding motion through the needle feeding support 3-3 fixedly connected therewith, and the head end protecting support 3-2 is installed on the steel wire guiding mechanism 2-1 to limit the maximum microneedle feeding displacement.

Such arrangement facilitates accurate delivery of the stepped shaft puncture needle 3-1. Other structure and composition are the same as those in any of specific embodiments 1-4.

Specific embodiment 6: the present embodiment is described in conjunction with FIG. 3 to FIG. 5, and the microneedle unit in the present embodiment comprises a stepped shaft puncture needle 3-1 and a Luer taper 3-6, wherein the Luer taper 3-6 is connected with the stepped shaft puncture needle 3-1, and the stepped shaft puncture needle 3-1 passes through the head end protecting support 3-2 and is connected with the head end protection support 3-2.

In such arrangement, both the head end and the tail end of each needle feeding module are equipped with a fixing device, which can not only provide protection for the stepped shaft puncture needle 3-1, but also maintain the feeding direction of a microneedle and improve the precision of operation. A direct drive scheme that the integrated driving motor is used in combination with the ball screw is adopted to further reduce the transmission error and ensure the high precision of puncture operation. Meanwhile, the tail end is designed with a standard interface which is fixed with the Luer taper, which can connect a variety of medical injection or suction equipment, accurately control the drug delivery flow and speed, and effectively solve the problem of microneedle tube blockage, facilitating disinfection treatment. Other composition and connection relationships are the same as those in any of specific embodiments 1-3.

Specific embodiment 7: the present embodiment is described in conjunction with FIG. 3 to FIG. 5, and the stepped shaft puncture needle 3-1 in the present embodiment is an integrated mechanism.

In such arrangement, the microneedle puncture mechanism adopts an integrated stepped shaft design to ensure high-precision puncture capability even in long range operation. Other composition and connection relationships are the same as those in any of specific embodiments 1-6.

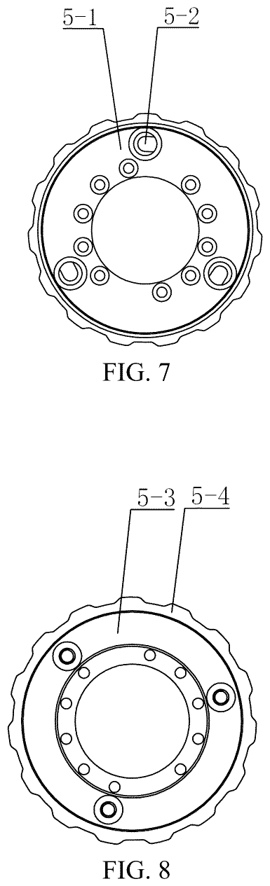

Specific embodiment 8: the present embodiment is described in conjunction with FIG. 3 to FIG. 5, FIG. 7 and FIG. 8, and the present embodiment also comprises a quick-change mounting mechanism 5, which is installed on the lower end surface of the high-precision linear driving module 4 and allows for quick attachment and detachment from an external robotic arm.

The quick-change mounting mechanism 5 in the present embodiment comprises a base 5-1, actuator positioning taper pins 5-2, a cover plate 5-3 and a lock piece 5-4, wherein the actuator positioning taper pins 5-2 are installed on the base 5-1, the lock piece 5-4 is installed on the outer circumference of the cover plate 5-3, and the cover plate 5-3 is buckled and connected with the base 5-1. When in use, the actuator can be installed and removed by rotating the quick-release locking piece clockwise with a wrench.

The specific implementation principle is as follows: the quick-release locking piece is rotated clockwise with the wrench by 25° to compress springs in three evenly spaced mounting holes around the circumference so that the three actuator positioning taper pins are removed from the elongated hole and align with the circular mounting hole, thus allowing the actuator to be automatically detached.

In the installation process, similarly, the locking piece is rotated clockwise with the wrench by 25° so that the lower circular hole is aligned with the positioning taper pins, then the positioning taper pins are inserted, and the springs are released to make the positioning taper pins return to the elongated hole limit position.

The quick-change device is fixed with the end joint of the robotic arm by standard distributed screws, and the actuator can be installed and removed by simply rotating the locking piece repeatedly. The precision of mechanical installation is ensured by the combination of the actuator positioning taper pins and the taper mounting holes of the quick-change base as well as the machining accuracy of the limiting hole of the quick-release locking piece, and the ball bearing design is adopted, which can play a role in smoothly rotating the locking piece to avoid motion jamming.

Specific embodiment 9: the present embodiment is described in conjunction with FIG. 2, and the present embodiment also comprises a split protective casing 6, wherein the split protective casing 6 is encased over the end-effector mechanism module 1, the driving and sensing module 2, the microneedle puncture mechanism module 3 and the high-precision linear driving module 4.

Such arrangement plays a role of disinfection and also protects the internal structure. Other composition and connection relationships are the same as those in any of specific embodiments 1-7.

Specific embodiment 10: the present embodiment is described in conjunction with FIG. 2, and the split protective casing 6 in the present embodiment comprises an actuating mechanism protective sleeve 6-1, an actuator casing 6-2 and a linear driving casing 6-3, wherein the actuating mechanism protective sleeve 6-1 is sheathed on the end-effector mechanism module 1, the actuator casing 6-2 is sheathed on the driving and sensing module 2 and the microneedle puncture mechanism module 3, and the linear driving casing 6-3 is sheathed on the high-precision linear driving module 4.

In such arrangement, the split casing in the present embodiment comprises three parts: the actuating mechanism protective sleeve 6-1 avoids contamination or damage to the instruments after disinfection and use; the actuator casing 6-2 adopts split design to reduce the difficulty of machining and assembly, the head end is made of metal to enhance the stability of the casing and facilitate cleaning and disinfection, and the tail end is designed with an end cover that can be opened and closed freely to facilitate internal wiring and maintenance; and the linear driving casing 6-3 protects the internal components and facilitates installation and maintenance. Such modular design not only simplifies assembly and maintenance, but also improves overall ease of use and reliability. Other composition and connection relationships are the same as those in any of specific embodiments 1-7.

The present invention adopts multi-source feedback information fusion in actual use:

In order to solve the problem that the surgical safety cannot be guaranteed due to insufficient state feedback during robot-assisted surgery, it is proposed that a variety of sensors are integrated in the actuator design, mainly comprising:

An endoscope module at the end of the continuum, with dimensions of only 0.65 mm*0.65 mm*1 mm, providing 400*400 first-view real-time intraoperative images and minimizing the hardware integration size on the premise of meeting the requirements of intraoperative images, which can help doctors judge the progress of the surgery, the target tissue location and the microneedle puncture state.

In addition, the tension sensor is integrated on the nut seat where the ball screw nut is placed to detect the tension state information of the steel wires during surgery. On one hand, the breakage of the steel wires caused by excessive tension during use can be effectively avoided, and the deformation of the steel wires can be compensated according to the real-time tension information to improve the control precision. On the other hand, whether the end-effector mechanism is in contact with the intra-ear environment can be judged according to the sudden tension changes to effectively ensure the safety of surgical operation.

Meanwhile, the driving device adopts an integrated scheme that the motors are used in combination with a gearbox and an encoder, and the encoder feedback can effectively ensure the accuracy and stability of the control signals of the drive input end, acquire the input displacement of stretching/release of each steel wire and the feeding depth of microneedle puncture, and feed back the intraoperative state information of the robot to improve the success rate of the surgery.

By fusing the information fed back by the above three types of sensors, more accurate and reliable intraoperative feedback based on multi-source information can be achieved:
1. Combined with real-time endoscope images and tension information, the interaction between the actuating mechanism and the intra-ear environment can be judged from the aspects such as whether the images and the signals of the force sensor have spike jitter.
2. Combined with tension information and motor encoder feedback, the compensation motion control for tension deformation of the steel wires can be achieved to further ensure the control precision of the robot.
3. During the operation of inner ear puncture injection, combined with endoscope images and motor control feedback, the state of puncture interaction between the microneedle and the inner ear round window can be identified to estimate the puncture depth and improve the success rate of surgical operation.

It should be noted that in order to facilitate system integration and size reduction, the above sensors all select the minimum size scheme available in the market that can meet the design requirements of the surgical actuator herein, and facilitate disinfection treatment.

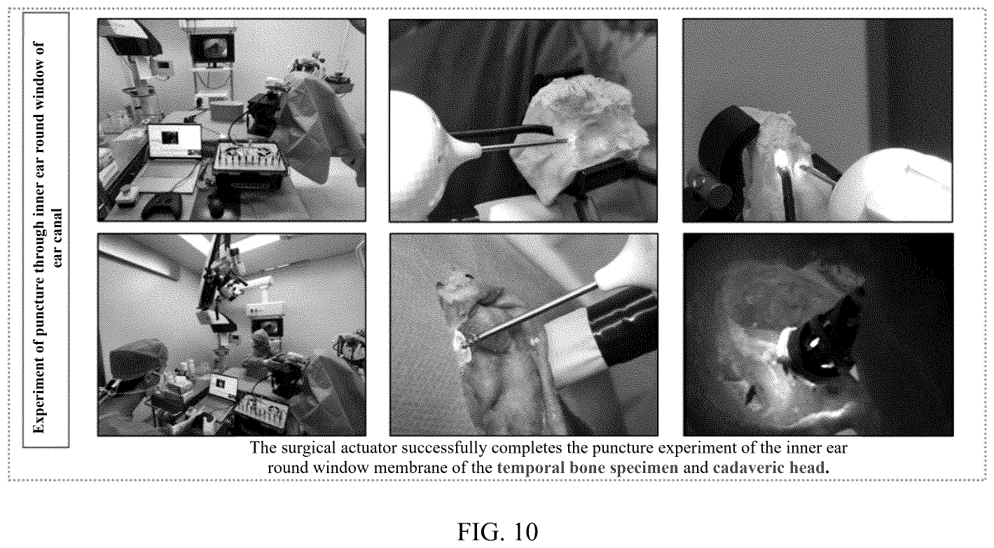

The working principle of the present invention is described in conjunction with FIG. 1 to FIG. 10:

The surgical actuator is installed on the universal cooperative robotic arm through the reserved interface of the quick-change mechanism to achieve accurate positioning, and the internal spring latch device can realize quick and stable installation and facilitate removal. By remote control, each motor receives control instructions and adjusts the displacement of the driving steel wires at micron resolution to achieve multi-degree-of-freedom motion of the end-effector mechanism. The endoscope provides real-time image feedback and guides the actuator to avoid structural obstacles in the intra-ear environment, and after reaching the target position, the flexible microneedle completes accurate puncture, injection and sampling operations. Combined with multi-source feedback signals such as images and tension, the doctors can adjust the operating parameters in real time to improve the precision and the success rate of the surgery and reduce potential injuries of the surgery to the inner ear.

The above embodiments are only used for describing the technical solution of the present invention rather than limiting the same. Although the present invention is described in detail by referring to the above embodiments, those ordinary skilled in the art should understand that the technical solution recorded in each of the above embodiments can be still amended, or some technical features therein can be replaced equivalently. However, these amendments or replacements do not enable the essence of the corresponding technical solution to depart from the spirit and the scope of the technical solution of various embodiments of the present invention.

What is claimed is:

1. A micro actuator for inner ear injection and sampling surgery, comprising an end-effector mechanism module (1), wherein the micro actuator also comprises a driving and sensing module (2), a microneedle puncture mechanism module (3) and a high-precision linear driving module (4);
the microneedle puncture mechanism module (3) is embedded in the driving and sensing module (2), the driving and sensing module (2) is installed on the high-precision linear driving module (4), the end-effector mechanism module (1) is installed in the front of the driving and sensing module (2), the microneedle puncture mechanism module (3) passes through the end-effector mechanism module (1) and extends, an endoscope module (1-2) located in the end-effector mechanism module (1) provides real-time images of a surgical process, and a tension sensor (2-2) in the driving and sensing module (2) monitors tension changes of steel wires in real time to ensure safe interaction with the intra-ear environment and provide early warning information when necessary.

2. The micro actuator for inner ear injection and sampling surgery according to claim 1, wherein the end-effector mechanism module (1) comprises a plurality of rolling saddle segments (1-1), an endoscope module (1-2) and a flexible microneedle (1-3);
the rolling saddle segments (1-1) are connected in series by multiple steel wires within the driving and sensing module (2), enabling multi-degree-of-freedom large-angle bending both in-plane and out-of-plane under the varying tension of each steel wire, the plurality of rolling saddle segments (1-1) form a hollow flexible structure, the endoscope module (1-2) and the flexible microneedle (1-3) are embedded inside the flexible structure, and the endoscope module (1-2) is located at the end of the flexible structure.

3. The micro actuator for inner ear injection and sampling surgery according to claim 2, wherein the driving and sensing module (2) comprises a plurality of driving units which are installed on a frame (2-7) in a ring array;
each driving unit comprises a steel wire guiding mechanism (2-1), a tension sensor (2-2), a signal amplifier (2-3), a micro guide rail (2-4), a steel wire driving motor (2-5), a steel wire (2-8) and an integrated driving board (2-6);
the steel wire guiding mechanism (2-1) is installed in the front of the frame (2-7), the steel wire driving motor (2-5) is installed in the back of the frame (2-7), the integrated driving board (2-6) is vertically installed at the rear end of the frame (2-7), and the micro guide rail (2-4) is installed in the middle of the frame (2-7);
the tension sensor (2-2) is fixedly installed on the micro guide rail (2-4) by a slider, the signal amplifier

(2-3) is installed above the tension sensor (2-2), one end of the steel wire (2-8) is connected with a threaded hole of the tension sensor (2-2) by a wire drawing device, and the other end of the steel wire (2-8) is fixed with the rolling saddle segments (1-1) after bypassing the steel wire guiding mechanism (2-1).

4. The micro actuator for inner ear injection and sampling surgery according to claim 3, wherein the microneedle puncture mechanism module (3) comprises a plurality of needle feeding modules and a stepped shaft microneedle unit, the plurality of needle feeding mechanisms are axially installed inside the frame (2-7), and the microneedle unit is coaxially installed in a central hole of the needle feeding mechanisms.

5. The micro actuator for inner ear injection and sampling surgery according to claim 4, wherein each needle feeding module comprises a head end protecting support (3-2), a needle feeding support (3-3), a microneedle puncture driving motor (3-4) and a tail end fixing device (3-5);
the needle feeding support (3-3) and the microneedle puncture driving motor (3-4) are installed on the frame (2-7) and centrally located inside the driving and sensing module (2), an output shaft of the microneedle puncture driving motor (3-4) drives a nut on a ball screw to make high-resolution linear motion and realize feeding motion through the needle feeding support (3-3) fixedly connected therewith, and the head end protecting support (3-2) is installed on the steel wire guiding mechanism (2-1) to limit the maximum microneedle feeding displacement.

6. The micro actuator for inner ear injection and sampling surgery according to claim 5, wherein the microneedle unit comprises a stepped shaft puncture needle (3-1) and a Luer taper (3-6), the Luer taper (3-6) is connected with the stepped shaft puncture needle (3-1), and the stepped shaft puncture needle (3-1) passes through the head end protecting support (3-2) and is connected with the head end protection support (3-2).

7. The micro actuator for inner ear injection and sampling surgery according to claim 6, wherein the stepped shaft puncture needle (3-1) is an integrated mechanism, capable of achieving precise puncturing with the end microneedle over a long transmission distance and meeting the pressure requirements for injection and sampling operations.

8. The micro actuator for inner ear injection and sampling surgery according to claim 7, also wherein it further includes a quick-change mounting mechanism (5), which is installed on the lower end surface of the high-precision linear driving module (4) and allows for quick attachment and detachment from an external robotic arm.

9. The micro actuator for inner ear injection and sampling surgery according to claim 1, wherein it further includes a split protective casing (6), which is encased over the end-effector mechanism module (1), the driving and sensing module (2), the microneedle puncture mechanism module (3) and the high-precision linear driving module (4).

10. The micro actuator for inner ear injection and sampling surgery according to claim 9, wherein the split protective casing (6) comprises an actuating mechanism protective sleeve (6-1), an actuator casing (6-2) and a linear driving casing (6-3), the actuating mechanism protective sleeve (6-1) is sheathed on the end-effector mechanism module (1), the actuator casing (6-2) is sheathed on the driving and sensing module (2) and the microneedle puncture mechanism module (3), and the linear driving casing (6-3) is sheathed on the high-precision linear driving module (4).
